(19) United States
(12) Patent Application Publication
(10) Pub. No.: US 2024/0423532 A1
(43) Pub. Date: Dec. 26, 2024

(54) SCREENING METHOD FOR IDENTIFICATION, AND TREATMENT PATHWAYS FOR SLEEP DISORDERS

(71) Applicant: Alvaro Sobalvarro Ruata, Guatemala City (GT)

(72) Inventor: Alvaro Sobalvarro Ruata, Guatemala City (GT)

(21) Appl. No.: 18/684,989

(22) PCT Filed: Sep. 30, 2021
(86) PCT No.: PCT/IB2021/059020

§ 371 Date: Feb. 20, 2024

(51) Int. Cl. A61B 5/00

(52) U.S. Cl. CPC A61B 5/4815; A61B 5/4818

(57) ABSTRACT

A method for etiologic screening of Sleep Breathing Disorders, the most common, Obstructive Sleep Apnea, that includes 6 steps, as following: (1) identifying patients cognitive capabilities, irritability and level of tiredness/fatigue at awakening, (2) day hypersomnia, (3) repetitive hypercaloric food-intake during the day and/or night, (4) extreme fatigue/tiredness in the afternoon-night, (5) snoring (OSA), (6) sleep fragmentation, poor sleep quality, night anxiety, night sweating, insomnia, etc. . . . In case of OSA, the sole responsible for such disturbance is the intermittent hypoxia, in which case, performing all the necessary adherence plans for the APAP is indicated.

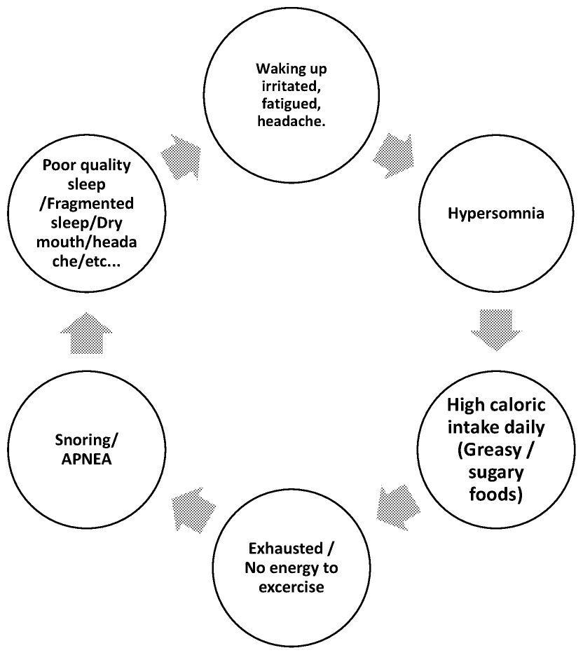

### FIELD OF INVENTION

The present invention relates to a screening and therapeutic indications for Sleep Breathing Disorders, Obstructive Sleep Apnea and/or any other sleep disorder that produces micro arousals.

### BACKGROUND OF THE INVENTION

Sleep Disorder Breathing disorders is common around the world. Obstructive Sleep Apnea (OSA) affects around 17% of world population (See S. Redline, et al. 2019; Sleep Disordered Breathing and Cardiac Disease) and in particular snoring, for a large part of the subjects involved. These patients are the mild, moderate, and heavy snorers, which are identified as “deep sleepers”.

Sleep Apnea is extremely underdiagnosed, being OSA the most common of all. OSA is responsible for 20% of all myocardial infarctions (250,000/year) and 15% of sudden cardiac deaths (47,500/year). This has serious effects on health care systems, being a massive underscore for cardiovascular events. (See Sharma et al. [2013], Impact of Treatment with Continouos Positive Airway Pressure (CPAP) on Weight in Obstructive Sleep Apnea)

Approximately 58% of patients with moderate to severe OSA are obese (Kimoff, n.d.). Up to now, obesity has been a direct cause of apnea due to the tissue deposition as well as to the lung volume-dependent effect. However, in most cases, indications of diet and exercise have proved not been able to provide any satisfactory solution. This is because of high expenditure of glucose (hepatic and muscular) during the night due to the intermittent hypoxemia, this in turn activates 2 specific nuclei in the brain (retrotrapezoid and locus coeruleus) which in turn liberate noradrenaline, and this produces all of its effects. (See Horner & Malhotra et. al (2016), Disorders of Sleep and Central Control of Breathing Ch. 85 at p. 1513)

Apnea occurs when an individual breathes very shallowly or stops breathing completely over a period of at least 10 seconds or more, resulting in a drop in blood oxygen level. Apneas usually occur during sleep and cause the individual to wake or transition from a deep level of sleep to a shallower sleep state. “Hypopneas” refer to decreases in breathing that also result in hypoxemia but are less severe than apneas. Generally, an apnea refers to a decrease in airflow or chest wall movement that is smaller than approximately 25% of baseline, while a hypopnea refers to a decrease of less than about 70% of baseline. See K. Banno et al. (2007) Sleep Medicine 8 (4): 400-426.

The International Classification of Sleep Disorders—2nd edition (ICDS-2) defines two categories of sleep-related breathing disorders, central sleep apnea syndrome (CSAS) and obstructive sleep apnea syndrome (OSAS). Mixed sleep apneas involve both CSAS and OSAS. The distinction between CSAS and OSAS relates to the mechanism that causes the respiratory disturbance. CSAS involves a dysfunction in ventilatory control in the central nervous system (CNS), with a reduction in impulses transmitted from the CNS to the respiratory muscles. OSAS, which is much more common than CSAS, is a disorder that is caused by physical obstruction of the upper airway. The obstruction typically results from abnormal control of the muscles that maintain the patency of the upper airway, and/or abnormal craniofacial anatomy. Common risk factors for OSAS include obesity, enlarged tonsils and adenoids, and craniofacial abnormalities. OSA should be deemed to have a continuum with the prevalence of 6-17% in the general adult population and 49% in the elderly population (with the OSA severity of apnea-hypopnea index_15) (Senaratna et al., 2017). The prevalence of OSA has been also increasing in parallel with the prevalence of obesity over the past two decades (Dempsey et al., 2010; Flegal et al., 2010; Memtsoudis et al., 2013; Peppard et al., 2013) (Wang et al., 2019)

OSAS has emerged as a common sleep disorder that is associated with excessive daytime sleepiness as well as more significant problems, including atherosclerosis, hypertension, heart failure, nocturnal cardiac arrhythmias, and an elevated risk of myocardial infarction and stroke. See, e.g., Sleep Apnea: Implications in Cardiovascular and Cerebrovascular Disease, 2 nd Ed., Bradley et al., eds. (Informa Healthcare USA, Inc., 2010) (particularly Levitzky et al., Ch. 10, at p. 163; Friedman et al., Ch. 11, at p. 180; Lorenzo-Filho et al., Ch. 13, at p. 219; Siccoli et al., Ch. 14, at p. 237; Sorajja et al., ch. 15, at p. 261; and Yumino et al., ch. 17, at p. 302). A diagnosis of OSAS is typically made when repetitive apnea or hypopnea events occur during sleep, with 5-15 episodes/hour classified as mild OSAS, 15-30 episodes/hour classified as moderate OSAS, and over 30episodes/hour classified as severe OSAS. Banno et al. (2007), citing Sleep-Related Breathing Disorders in Adults: Recommendations for Syndrome Definition and Measurement Techniques in Clinical Research, in Report of an American Academy of Sleep Medicine Task Force (1999), Sleep 22 (5): 667-689.

Normally the diagnosis of the OSAS is made with the to use devices to determine if the patients suffer from this disorder, examples of the state art are the patent applications WO2011082199, CN248909404, CN192975515, WO2012052882, US20040225226, etc. the present invention is a very practical and simple method to diagnosis the OSAS without the use of devices or complex chemical or physical analysis.

OSAS is commonly treated using the Automatic Positive Air Pressure (APAP) technique, in which a continuous and automatic stream of compressed air is administered to the patient using a machine specifically designed for that purpose. Other forms of treatment include intraoral mandibular advancement devices and craniofacial surgery. These methods are cumbersome and expensive, and although many pharmacological agents have been proposed and evaluated, no agent has proved to be successful in treating OSAS.

### OBJECT AND SUMMARY OF THE INVENTION

The present invention addresses the need in the art and provides screening method for Sleep Disorder Breathing, being Obstructive Sleep Apnea (OSA) the most common.

By “OSAS,” as the term is used herein, applicants are referring to obstructive sleep apnea syndrome as defined above, but do not intend to exclude the possibility that the individual being treated may also have some degree of Central Sleep Apnea Syndrome (CSAS) or Restless Leg Syndrome (RLS)/Periodic Limb Movement Disorder (PLMS).

In addition, by “treating OSAS” applicants are referring to (1) the elimination of nighttime apneas and/or hypopneas, (2) a reduction in the number of apneas and/or hypopneas per hour and/or per night, and/or (3) the amelioration of the extent of each apnea and/or hypopnea event experienced by the individual undergoing treatment, the APAP (as may be determined, for instance, by an increase in airflow or in the amplitude of chest wall movement).

In one aspect, then, the invention provides a method for screening OSAS by describing the next signs/symptoms:
1. Waking up irritable/cranky/restless (tired)
2. Day hypersomnia, patient tends to fall asleep during the day, in any type of situation.
3. Hypercaloric intake during the day and/or night
4. Fatigue/tiredness in the afternoon/evening, patient just wants to go to sleep, due to the latter, no exercise.
5. Snoring (Apnea) starts within the initiation and maintains itself during the sleeping period. This in turn produces fragmented sleep.
6. Fragmented sleep induces micro-arousals, arousals, anxiety, palpitations, diaphoresis, etc.
6a. The APAP treatment should start here, this for preventing every single direct adverse effect the intermittent hypoxia induces (all being previously mentioned) and for the patient to have a good quality of life, or the most “normal” life possible.
7. This produces an all-around poor quality of sleep and an extremely poor quality of life.

In a further aspect, the invention provides a method for treating OSAS in a patient by co-administering:
(a) A therapeutically effective amount of benzodiazepines and non-benzodiazepines
(b) This may be in the patients that present early psychological rejection and/or anxiety due to the claustrophobia of using a mask.
(c) This should be administered in conjunction with the APAP until the patient has been adapted (˜3 to 8 weeks is the average time patients take to adapt to the APAP, especially the mask) and it should be discussed with the patient if they want to withdrawal from the use of the latter, if not, it should be continued until new notice.

Waking up irritable/cranky/headache, patient goes through the day with a bad attitude, cognitive functions are on the lowest, this in turn makes the patient make mistakes involuntarily, due to the brain trying to get some rest. This is due to the awakenings (micro-arousals) during the night, in this case due to Obstructive Sleep Apnea (OSA). This produces fragmented sleep; this produces a constant awakening (without the patient being able to notice) and the brain does not get enough restorative sleep (stage N3).

Hypersomnia (during the day), patient goes through the day with a high probability of falling and sleep and a high tendency of sleepiness, this is due to the awakenings (micro-arousals) during the night, in this case due to Obstructive Sleep Apnea (OSA). This produces fragmented sleep; this produces constant awakenings (without the patient being able to notice) and the brain does not get enough restorative sleep (stage N3).

High intake of hypercaloric foods, repetitively during the day and/or night. Patient is always “anxious/craving” about greasy, starchy and/or sugary food, to compensate the consumption of glucose during the night. This is explained via the repetitive and extreme muscular effort to breathe during the night, due to the intermittent hypoxia due to the Obstructive Sleep Apnea (OSA). Patient with overweight/obese (BMI: 28 kg/m²), hypertension (BP≥130/90 mmHg, “dipping pattern”, mostly HBP during the night), insomnia, anxiety attacks abruptly in the middle of the night, palpitations in the middle of the night, night terrors, dry mouth/throat at awakening, headache/migraine at awakening are the ones with highest risk of presenting this “symptom”.

Extreme exhaustion/fatigue in the night, does not want to perform any type of exercise. Patient just wants to go to sleep and have a restful night. In this phase, “anxiety of going to sleep” kicks in, patient starts presenting insomnia or fear of going to sleep, this is a defense mechanism that the brain induces, this is performed so the body does not suffer the extremely injurious effects of the intermittent hypoxia. Patients present with nocturnal myocardial infarction, nocturnal hypertension, arrythmias, tachycardia, etc.

Snoring (in most patients, some may NOT snore and still present obstructive sleep apnea and/or intermittent hypoxia), patient falls asleep, the “snoring” initiates and the intermittent hypoxia is the result. Patients present with turbine hypertrophy, deviated septum, Mallampati score (≥2), smokers, hypertrophy of the base of the tongue, hypertrophy of the uvula and soft palate (due to the vibration produced by the snoring and this induces a chronic inflammation), micrognathia and/or retrognathia, craniofacial deformities/anomalies (Marfan, Acromegalia, etc.), tumors in the Upper Airway area.

Fragmented sleep: micro-arousals, awakenings with palpitations, anxiety, extremely poor sleep quality, tachycardia, “pounding headache”, dry mouth (may be mild to severe, this means, patient wakes up just to drink water due to dryness), dry throat (discomfort of swallowing; dysphagia in some cases), awakenings in the middle of the night associated with extreme irritability and insomnia after this, tendency to eat (hypercaloric foods).

The steps are:
1. Irritability at awakening, feeling unrested/fatigued, cranky, dry mouth/throat (dysphagia in some cases), headache (migraine type) at awakening.
2. Hypersomnia, high tendency to sleep during the day. Patient may fall asleep in the bathroom, classroom, driving, movies, etc.
3. Hypercaloric intake during the day. Patient has an “uncontrollable” tendency for the intake of hypercaloric foods (starchy, sugary and/or fatty foods). Some patients may have the tendency to eat something sweet/fatty before bedtime.
4. Exhaustion/fatigued in the night. Patient is extremely tired, just wants to sleep. No physical activity will the patient perform, nor will his body let him as well.
5. Snoring/Obstructive sleep apnea initiates, this in turn induces chronic intermittent hypoxia.
6. Fragmented sleep, this is a direct effect of the intermittent hypoxia which produces micro-arousals.

Furthermore, the steps for identifying the signs responsible for the Obstructive Sleep Apnea (OSA), may compromise the complete evaluation of the patient. This is because all the above signs are a direct consequence of intermittent hypoxia due to, the collapse of the upper airway, this traduces to Obstructive Sleep Apnea. For this reason, the examiner must complete the full evaluation and follow step by step the previous cycle.

The screening method may also provide for the investigation of hypertension, arrythmias, insulin resistance, Diabetes Mellitus II, metabolic syndrome, and/or sleep disorders associated with micro-arousals (Example: Restless Leg Syndrome/Periodic Limb Movement Disorder).

According to the same inventive concept, the invention also provides therapeutic indications for possible treatments, this includes CPAP/APAP, BiPAP and/or surgery. Compromising the “snoring” as the responsible for the patients Obstructive Sleep Apnea and/or symptoms present.

The present invention also provides a screening method for other etiologic disturbances/pathologies:
Identifying the type of Hypertension, specifically nocturnal. Holter has nocturnal spikes and normalization of the blood pressure during the day.
Identifying nocturnal hyperglycemia. This can be interpreted as insulin resistance.
Identifying nocturnal arrythmias.
Identifying “panic attacks” in the middle of the night, associated with insomnia and anxiety during the day. Patient is diagnosed with anxiety.
Identifying nocturnal hidrosis and hyperhidrosis.
Identifying morning headaches/migraine that normalize during the day or diminishes.

In the present context, the expression of micro-arousals “related” sleep disorders is to be understood in a wide sense, compromising all the symptoms/signs secondary to the latter. This includes most of the respiratory anomalies during sleep, obesity as a sign and as a direct adverse effect, and all the metabolic syndrome signs/symptoms, this eventually is associated with Sleep Breathing Disorders, and the complications surrounding sleep due to the intermittent hypoxia and/or the intermittent secretion of catecholamines as a direct result of micro arousals.

The present invention provides relevant advantages; the main advantage lies in the fact that the screening method allows reliable functional screening to be carried out in any clinical setting or anywhere and/or with any type of computer, tablet, smartphone, smartwatch or any technological smart device for the screening/diagnosis and treatment for Sleep Breathing Disorders. The screening method of the invention allows functional screening without the need to prescribe pharmacological treatment (and this can be extremely dangerous to this type of patients) and perform the screening correctly once all the steps have been filled out. This will give the practitioner a fast and assertive way to assess if the patient needs to perform a sleep study and confirm the diagnosis.

### DETAILED DESCRIPTION

The screening method of the invention moves away from observation. In particular, as explained in detail in the following, a clinical study was carried out showing that patients with Obstructive Sleep Apnea associated with fragmented sleep (micro arousals due to the intermittent hypoxia) have a tendency to crave sugary/starchy and fatty/greasy foods. (Beebe et al., 2011, page 1)

More specifically, this study showed that obesity was a major cause of sleep apnea. On the contrary, Obstructive Sleep Apnea (OSA) associated with intermittent hypoxia is a major cause, if not the most important cause of obesity, due to the intense necessity of eating highly hypercaloric foods to compensate for the consumption of glucose during the night due to the hypoxia induced by Obstructive Sleep Apnea.

Due to the metabolic syndrome, this is mostly due to the intermittent hypoxia induce by Obstructive Sleep Apnea, the real issue is the intermittent secretion of catecholamines, in theory, in every episode of hypoxia. This induces a catabolic state, and the patient utilizes more energy asleep than awake. In this specific event, patients always or the vast majority of time tend to start ingesting hypercaloric foods (carbonated drinks, deep fried foods, energy drinks, etc.), due to the high intake, and extreme and chronic fatigue, the patient continues in this pathway and eventually becomes overweight, then obese as an adverse effect and the final result. (Horner & Malhotra, 2016)

Accordingly, in studies, it's said that the laryngeal, oropharyngeal, nasopharyngeal muscles are obliterated, also all the other muscles in the upper airway. This area (nasopharyngeal, oropharyngeal, and laryngeal) does not function properly, the muscular tone is lost, and the muscles tend to collapse inwardly, mostly during REM sleep (due to glycine and galanin) therefore the collapse of the upper airway occurs (obstructive sleep apnea), and the intermittent hypoxia initiates.

As mentioned above, due to such improper operation of the Central Nervous System, in REM sleep the secretion of Galanin and Glycine (this produces a complete spinal motor activity suppression), for this reason a complete relaxation of the muscles in the body occurs, the area of interest is the Upper Airway, which includes, pharyngeal, palatine and tongue muscles, this in turn produces a complete or partial collapse of the latter, that induces the obstructive sleep apnea, and therefore the intermittent hypoxia. When this happens, the retrotrapezoid nucleus and the locus coeruleus are activated, and the latter secretes norepinephrine in an asynchronous way, this has a relative function of producing a generalized muscle contraction, this is for the area of the upper airway that is collapsed, can regain its tone, and the patient may start breathing.

The above observations therefore indicate that there is no bone/rigid structure in that space of the upper airway, but it has numerous sensory areas.

The muscles that are responsible for maintaining the upper airway open are:
Genioglossus
Geniohyoid
Tensor and levator velo palatini
Pharyngeal constrictors

On the basis of the above considerations, the general inventive concept of the present invention lies in the fact that the Upper Airway muscles/regions from which the normal function is to maintain a permeable airway so that the airflow may pass in and out. This function depends on multiple factors including sleep stages, neurotransmitters (Galanin and Glycine) which in turn the brain produces a wide variety of symptoms to alert the patient that his breathing is irregular during sleep.

A specific example of implementation of screening method of the invention will now be described.

According to the first step of the screening method, the type of awakening, humor (irritated), attitude and fatigued (more than the day before, even though the patient “slept”) with one or more awakenings at night.

Example: Irritability when waking up, most of the time associated with headache and/or dry mouth and throat, sometimes halitosis, not knowing why the patient is unable to restore sleep even though the patient slept.

Second step, hypersomnia, tendency to sleep or fall asleep during the day.

Example: Patient will fall asleep involuntarily in waiting rooms, talking with someone else, seeing a movie, work-meeting, etc.

The screening method just described may be used on any sleep disorder that causes excessive daytime sleepiness with hypercaloric intake and it must be associated with micro arousals.

In particular, this second step, relates to the direct effect of the sixth step (micro arousals), they induce a pulsatile secretion of catecholamines (noradrenaline), this in turn consumes glycogen (hepatic and muscular) during the night, for that reason the patient presents with excessive daytime sleepiness (EDS). This induces a change in the eating habits, and the patient has an urge/necessity to ingest hypercaloric foods (sugary, starchy and/or fatty foods).

Third step, hypercaloric intake multiple times during the day, this happens for the excessive fatigue/tiredness the patient presents during the day, this is due because of the process of fragmented sleep, it consumes more energy during sleep than during wakefulness. Patient is constantly looking for sugary/fatty foods to compensate the loss of energy during the night. This can be correlated with the Index of Awakenings.

Fourth step, extreme fatigue/tiredness at evening/night, no energy to perform physical activity whatsoever. Patient probably will wake up multiple times around 2 or 3 am. An article described in which process does the obstructive sleep apnea process insomnia. (Sweetman et al., 2017) (Sweetman et al., 2017)

Fifth step, snoring, that converts into obstructive sleep apnea, and this therefore, intermittent hypoxia.

Sixth step, fragmented sleep, poor quality of sleep, insomnia, anxiety, palpitations, irritability, and depression.

The present invention relates to an integrated sleep screening, and more particularly to an integrated apnea screening method. The present invention additionally relates to methods of sleep screening. The sleep disorder treatment system of the present invention can use this diagnosis screening method to analyze various characteristics (physical and physiological) of each patient to determine of subject's sleeping disorder or symptoms of a subject's sleep disorder. The screening method uses many different types of laboratory test, physical findings, and specific behavior characteristics of each patient, for screening the severity of the symptoms of the sleep disorder itself.

This is a method for secure screening for sleep apnea for the general population and obese population as well. The present invention has been hereto describing the process by which obstructive sleep apnea causes obesity or is a major cause due to the intermittent hypoxia. This screening method allows reliable functional screening to be carried out in any clinical setting or anywhere.

### EXAMPLES

Various techniques known to those in the art and/or described in the pertinent literature and texts can be implemented to demonstrate the efficacy of the present combinations in the treatment of OSA.

Male patient was evaluated, which was obese, type II diabetic, previous PSG and AHI≥30/hr, heavy snorer. This patient was started on Automatic Positive Airway Pressure (APAP) as a first line treatment and suggested interconsult with an ENT and a Nutritionist. Evaluated every 2 weeks, patients presented with a reduction in sugary/starchy/fatty foods, and this effect led to the weight loss expected, approximately 1-2 pounds per week. After 3 months, patient was compliant with the APAP, energetic and started to perform exercise 3 times per week. Patient started with the nutritionist plan, and at the end of the 6 months, patient lost 15 pounds. This patient was compliant, had a very good response to APAP, due to the induction received and the multidisciplinary team that intervened. Patient underwent a strict control with his Diabetologist during the 6 months, he started treatment with Glisulin 1000 mg and short acting insulin on sliding scale before each meal. Due to the APAP intervention, insulin doses were markedly reduced.

Female patient, 54, morbid obesity, with ventricular tachycardia, heavy snorer, she refers that she acquired a smartwatch (newest version with saturation indicator), she started monitoring her night saturation. The monitoring of 7 nights was an oxygen saturation average of 84%, because of this, she started investigating on her laptop (Windows, Surface 6) online, which in turn lead her to the Sleep Clinic. Patient made an appointment; she was asked to fill the medical history and at the end, the sleep tech passed the screening method using a tablet and scored 5 of 6. With this score, the sleep specialist suggested she should perform an In-Lab Polysomnogram (PSG). The result was positive for Severe Obstructive Sleep Apnea (AHI: 63) associated with severe hypoxemia (84minutes during the PSG).

Male patient, 34 years old, BMI 24 kg/m², athlete (marathon runner), with insomnia of 1 year, at 2 am, wakes up gasping and loud snoring. Bedpartner complains. Patient starts web-searching on his smartphone and finds the screening method, clicks on the link, patient scores 4 of 6. (Those 4 points were: snoring, irritability, hypersomnia, and hyper caloric intake of sugary foods.) This result suggest he may have a sleep disorder, so he decides to make an appointment in a Sleep Clinic. The Somnologist analyses the data from the screening method, his physical exam and symptoms, so he suggests an In Lab Polysomnography (PSG), which the patient performs, and the result was, Mild Obstructive Sleep Apnea with an Elevated Awakening Index (AWI: 24; this means 24 micro-arousals per hour of sleep) associated with severe snoring.

Female patient, 19 years old, university student, BMI: 19 kg/m², snorer and daily hypersomnia, teacher noticed she was always asleep during class. Teacher used his tablet and started searching on the internet for the symptoms. He was redirected to the vicious cycle, which he re sent to the student. Student filled out the steps, she scored 3 of 6. The teacher suggested her to see a Sleep Specialist. She performed a Home Sleep Apnea Test; the result was positive for Mild Obstructive Sleep Apnea associated with severe hypoxemia (Spent 80 min with saturation below 90%). Patient was referred to an Otorhinolaryngologist for an evaluation, which she had a Mallampathi class IV, deviated septum and turbinate hypertrophy. She performed surgery 2 weeks after. She performed an Home Sleep Apnea Test again, which was normal for obstructive sleep apnea, no snoring, no hypoxemia during the study.

1. A method for etiologic screening of a sleep disorder associated with micro-arousals comprising:
identifying, in a first step, at least one of the following symptoms in a patient: Irritability, bad attitude, headache at awakening of the patient, dry mouth or throat at the awakening of the patient, anxiety, or heart palpitations while sleeping;
identifying, in a second step, if the patient has hypersomnia during the day that, wherein hypersomnia is >8 on the Epworth sleepiness scale;
identifying, in a third step, if the patient has repetitive hypercaloric intake during the day due to chronic fatigue and exhaustion induced by the sleep disorder induces and if the patient has a BMI>28 kg/m2;
identifying, in a fourth step, if the patient experiences exhaustion or fatigue in the afternoon or night;
identifying, in a fifth step, if the patient experiences snoring that converts into obstructive sleep apnea; and
identifying, in a sixth step, if the patient experiences fragmented sleep with an awakening index>8.

2-5. (canceled).

6. The method according to claim 1, further comprising:
identifying, in the third step, if the patient has repetitive hypercaloric intake by also identifying if the patient experiences an urge or tendency to ingest hypercaloric foods.

7-10. (canceled).

11. The method according to claim 1, further comprising:
treating the sleep disorder associated with micro-arousals by administering a therapeutically effective amount of benzodiazepines and nonbenzodiazepines.

12. The method according to claim 11, further comprising:
treating the sleep disorder associated with micro-arousals by co-administering an automatic positive airway pressure (APAP) using a mask on the patient for at least 3 to 8 weeks.

13. The method according to claim 11, further comprising:
utilizing a computing device for the identification in the first step, the second step, the third step, the fourth step, the fifth step, and the sixth step.

14. The method according to claim 11, further comprising:
treating the obstructive sleep apnea with a positive pressure device.
